(19) United States
(12) Patent Application Publication
(10) Pub. No.: US 2026/0248426 A1
(43) Pub. Date: Aug. 27, 2026

(54) INFORMATION PROCESSING APPARATUS, INFORMATION PROCESSING METHOD, AND NON-TRANSITORY COMPUTER-READABLE MEDIUM

(71) Applicant: NEC Corporation, Minato-ku, Tokyo (JP)

(72) Inventor: Sojung CHOI, Tokyo (JP)

(73) Assignee: NEC Corporation, Minato-ku, Tokyo (JP)

(21) Appl. No.: 18/875,844

(22) PCT Filed: Jul. 14, 2022
(86) PCT No.: PCT/JP2022/027734

§ 371 Date: Dec. 17, 2024

(51) Int. Cl. A61B 5/16

(52) U.S. Cl. CPC A61B 5/165

(57) ABSTRACT

An information processing apparatus (100) includes a computation unit (102) that processes biometric information of a subject, and thereby computes a score indicating a mental state of the subject, an estimation unit (104) that estimates an emotion of the subject by use of a criterion including a threshold value associated with the subject, and the score, and an update unit (106) that updates the threshold value by use of a history of the score.

### TECHNICAL FIELD

The present invention relates to an information processing apparatus, an information processing method, and a program medium.

### BACKGROUND ART

There is a system that processes voice and an expression of a subject by use of voice or image recognition, and analyzes an emotion of the subject. Patent Document 1 discloses an apparatus that processes an image capturing an expression of a user operating an image formation apparatus such as a camera, decides whether the user is confused, and displays a help image in a case where the user is decided to be confused. The Patent Document 1 describes a contrivance of solving accuracy deterioration of emotion estimation due to an individual difference in a rising degree of a corner of a mouth of a face of a person or a falling degree of an eyebrow. In order to solve this problem, the image formation apparatus described in Patent Document 1 is an apparatus that captures a criterion face that is to be used as a criterion for an expression change, and a decision subject face, as face images of a user, decides an emotion in the decision subject face, based on a change amount in the expression change from the criterion face, and performs message support being relevant to an emotion in the decision subject face, and the image formation apparatus includes a detection unit that detects an input operation performed by a user without stagnation in setting of a print condition at execution of a print job by a request of the user, and an acquisition unit that acquires, as a criterion face, the face image captured by a capture unit, at a time point when the input operation without stagnation is detected by the detection unit, and an expression of the user is assumed to become a criterion for an expression change.

Patent Document 2 describes an apparatus that recognizes individuality of a user, determines an individual, and improves recognition accuracy in a case of performing emotion recognition of the user. Patent Document 2 describes a contrivance of reducing such erroneous recognition that a user with a slanted eye is given emotion recognition of “being angry” emotion even though the user is not angry. In the apparatus described in Patent Document 2, an individuality recognition unit compares a feature value of the user extracted by each of a voice recognition unit, an image recognition unit, and a motion recognition unit with data for individual determination stored in an individuality DB, recognizes individuality of the user, determines an individual, reads an individuality data group being relevant to the determined user, and outputs the read individuality data group to each emotion recognition unit. Each emotion recognition unit performs emotion recognition by use of data stored in a voice DB, an image DB, and a motion DB, based on a difference between a feature value of the user extracted by each of the voice recognition unit, the image recognition unit, and the motion recognition unit, and the individuality data group given from the individuality recognition unit.

### RELATED DOCUMENTS

### Patent Documents

Patent Document 1: Japanese Patent Application Publication No. 2020-163660
Patent Document 2: Japanese Patent Application Publication No. 2001-83984

### SUMMARY OF INVENTION

### Technical Problem

A technique described in each of the above documents performs emotion estimation of a user in a case of being specialized for a situation such as a case of operating an image formation apparatus or driving a car, and therefore improves accuracy of emotion estimation of the user in this case. Thus, the technique described in the above document does not assume automatically adjusting an individual difference for each subject even though an employee is replaced, which is requested in a such a case of intending to perform emotion estimation of stress, anxiety, and the like of an employee in a workplace where personnel are replaced, and improve workplace environment. In these circumstances, the present inventor has considered automatically adjusting an individual difference for each subject even in a workplace where personnel are replaced, and improving emotion estimation accuracy according to individuality of each employee being a subject.

In view of the problem described above, one example of an object of the present invention is to provide an information processing apparatus, an information processing method, and a program medium that solve accuracy deterioration due to an individual difference in biometric information in an emotion analysis using biometric information.

### Solution to Problem

According to one aspect of the present invention, there is provided an information processing apparatus including:
a computation unit that processes biometric information of a subject, and thereby computes a score indicating a mental state of the subject;
an estimation unit that estimates an emotion of the subject by use of a criterion including a threshold value associated with the subject, and the score; and
an update unit that updates the threshold value by use of a history of the score.

According to one aspect of the present invention, there is provided an information processing method including,
by one or more computers:
processing biometric information of a subject, and thereby computing a score indicating a mental state of the subject;
estimating an emotion of the subject by use of a criterion including a threshold value associated with the subject, and the score; and
updating the threshold value by use of a history of the score.

According to one aspect of the present invention, there is provided a computer-readable program medium storing a program for causing a computer to execute:
a procedure of processing biometric information of a subject, and thereby computing a score indicating a mental state of the subject;
a procedure of estimating an emotion of the subject by use of a criterion including a threshold value associated with the subject, and the score; and
a procedure of updating the threshold value by use of a history of the score.

### Advantageous Effects of Invention

According to one aspect of the present invention, an information processing apparatus, an information processing method, and a program medium that can consider an individual difference in biometric information and improve accuracy in an emotion analysis using biometric information can be acquired.

### BRIEF DESCRIPTION OF THE DRAWINGS

FIG. 1 It is a diagram illustrating an outline of an information processing apparatus according to an example embodiment.

FIG. 2 It is a flowchart illustrating an operation example of the information processing apparatus according to the example embodiment.

FIG. 3 It is a diagram conceptually illustrating a system configuration of an information processing system according to the example embodiment.

FIG. 4 It is a block diagram illustrating a hardware configuration of a computer that implements the information processing apparatus.

FIG. 5 It is a functional block diagram illustrating a logical configuration example of the information processing apparatus according to the example embodiment.

FIG. 6 It is a diagram illustrating a data structure example of biometric information.

FIG. 7 It is a diagram illustrating a data structure example of score history information.

FIG. 8 It is a diagram for describing an emotion estimation method using an arousal level and a harmony level.

FIG. 9 It is a diagram for describing an individual difference in biometric information and adjustment of a threshold value.

FIG. 10 It is a diagram for describing an emotion analysis using a threshold value updated by an update unit.

FIG. 11 It is a diagram illustrating a data structure example of threshold value information.

FIG. 12 It is a flowchart illustrating an operation example of the information processing apparatus according to the example embodiment.

FIG. 13 It is a diagram for describing adjustment of a threshold value.

FIG. 14 It is a diagram for describing adjustment of a threshold value.

FIG. 15 It is a diagram conceptually illustrating a system configuration of the information processing system according to the example embodiment.

FIG. 16 It is a diagram illustrating an outline of the information processing apparatus according to the example embodiment.

FIG. 17 It is a diagram conceptually illustrating a system configuration of the information processing system according to the example embodiment.

FIG. 18 It is a functional block diagram illustrating a functional configuration example of the information processing apparatus according to the example embodiment.

FIG. 19 It is a flowchart illustrating an operation example of the information processing apparatus according to the example embodiment.

FIG. 20 It is a diagram conceptually illustrating a system configuration of the information processing system according to the example embodiment.

FIG. 21 It is a diagram illustrating an outline of the information processing apparatus according to the example embodiment.

### EXAMPLE EMBODIMENT

Hereinafter, example embodiments according to the present invention are described by use of the drawings. Note that, in all the drawings, a similar component is assigned with a similar reference sign, and description thereof is not included as appropriate. Moreover, in each of the following figures, a configuration of a part that is not related to an essence of the present invention is not included and not illustrated.

In the example embodiment, “acquisition” includes at least one of fetching, by a local apparatus, data or information stored in another apparatus or a storage medium (active acquisition), and inputting, into a local apparatus, data or information output from another apparatus (passive acquisition). Examples of active acquisition include requesting or inquiring of the another apparatus and receiving a reply thereof, accessing the another apparatus or the storage medium and reading, and the like. Moreover, an example of passive acquisition includes receiving information given by distribution (or transmission, push notification, or the like), and the like. Further, “acquisition” may include selecting and acquiring from received data or information, or selecting and receiving distributed data or information.

### Minimum Configuration Example

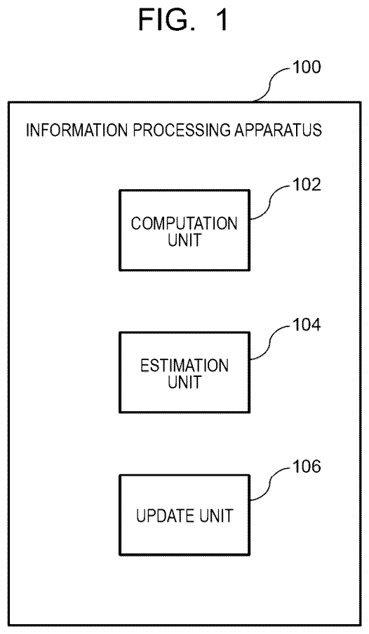

FIG. 1 is a diagram illustrating an outline of an information processing apparatus 100 according to an example embodiment. The information processing apparatus 100 includes a computation unit 102, an estimation unit 104, and an update unit 106.

The computation unit 102 processes biometric information of a subject, and thereby computes a score indicating a mental state of the subject.

The estimation unit 104 estimates an emotion of the subject by use of the score and a criterion including a threshold value associated with the subject.

The update unit 106 updates the threshold value by use of a history of the score.

### Operation Example

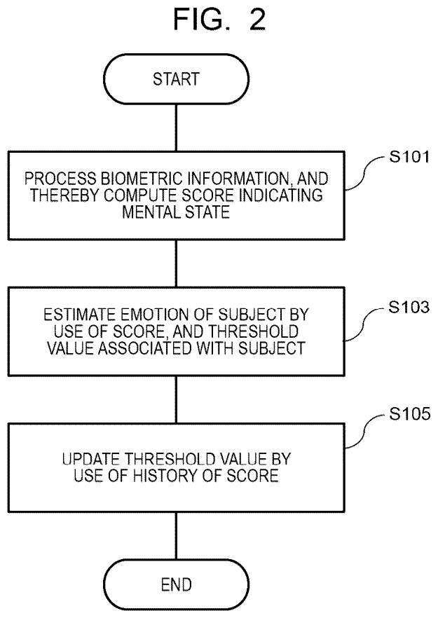

FIG. 2 is a flowchart illustrating an operation example of the information processing apparatus 100 according to the example embodiment.

The computation unit 102 processes biometric information of a subject, and thereby computes a score indicating a mental state of the subject (step S101).

The estimation unit 104 estimates an emotion of the subject by use of the score and a criterion including a threshold value associated with the subject (step S103).

The update unit 106 updates the threshold value by use of a history of the score (step S105).

Steps S103 and S105 of the present processing may be executed, for example, every time biometric information of a subject is acquired, or may be executed periodically at a predetermined timing. The predetermined timing is, for example, every hour, every half day, every day, or every week. Then, step S105 may be executed, for example, periodically at another predetermined timing. Another predetermined timing is, for example, every week, every other week, or every month, or the like. For a score history, history information accumulated between an execution timing before a current execution timing and the current execution timing is used.

In this information processing apparatus 100, the computation unit 102 processes biometric information of a subject, and thereby computes a score indicating a mental state of the subject. The estimation unit 104 estimates an emotion of the subject by use of the score and a criterion including a threshold value associated with the subject. The update unit 106 updates the threshold value by use of a history of the score.

In this way, the information processing apparatus 100 can consider an individual difference in biometric information and improve accuracy in an emotion analysis using biometric information.

A detailed example of the information processing apparatus 100 is described below.

### First Example Embodiment

### System Outline

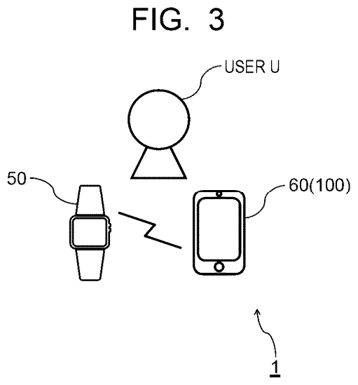

FIG. 3 is a diagram conceptually illustrating a system configuration of the information processing system 1 according to the example embodiment.

The information processing system 1 includes an information processing apparatus 100 and a wearable terminal 50.

In this example, the information processing apparatus 100 is a user terminal 60 of a user U, and is, for example, a computer such as a smartphone, a tablet terminal, or a personal computer. A pairing procedure is performed in advance for the user terminal 60 and the wearable terminal 50 in such a way that communication using near field communication (NFC) becomes possible.

In the information processing system 1, the user U is determined to be a subject of an emotion analysis, and the user U is, for example, an employee of a company. In this example, the user terminal 60 is owned by the user U, and the wearable terminal 50 is lent to user U from a company. However, the user terminal 60 may not necessarily be owned by the user U, and may be lent to the user U from a company. Moreover, the wearable terminal 50 may not necessarily be lent to the user U from a company, and may be owned by the user U.

Herein, since the wearable terminal 50 is lent to the user U from a company, the user U borrows the wearable terminal 50 when going to work, performs pairing of the wearable terminal 50 and the user terminal 60, wears the wearable terminal 50, engages in work, cancels pairing of the wearable terminal 50 and the user terminal 60 when leaving work, and returns the wearable terminal 50. That is to say, the wearable terminal 50 can be shared and utilized among employees. Thus, in a case where a work pattern is a shift system, the wearable terminals 50 may be prepared not for the number of employees but for the number of people present in a day.

The wearable terminal 50 measures biometric information of the user U, and transmits the biometric information to the user terminal 60. The biometric information acquired by the wearable terminal 50 includes various pieces of biometric information such as a pulse rate, a heart rate, a body temperature, blood pressure, an activity amount, a pose, an electrocardiogram, a respiratory rate, a blood oxygen level, and conversation voice, and is not limited to thereto.

The wearable terminal 50 is equipped with various sensors for measuring the pieces of biometric information. Moreover, regarding conversational voice, voice collected from a speaker of the wearable terminal 50 may be recorded, and voice data may be transmitted to the user terminal 60. The user terminal 60 side computes a conversation amount by processing the voice data, and records the conversation amount as biometric information of the user U. However, the wearable terminal 50 side may process the voice data, and transmit a conversation amount to the user terminal 60. Note that the conversation amount does not include information relating to a content of a conversation. The conversation amount can be indicated by, for example, the number of words uttered per time unit, or the like. Moreover, in an example embodiment described later, in a configuration including a server apparatus 70, voice data may be transmitted to the server apparatus 70, a conversation amount may be computed by processing the voice data on the server apparatus 70 side, and biometric information of the user U may be generated.

An application for utilizing a service of the present information processing system 1 is previously installed on the user terminal 60 of the user U. The user U wears wearable terminal 50, starts an application on the user terminal 60, and performs pairing of the wearable terminal 50 and the user terminal 60. Thereby, the information processing apparatus 100 can implement a function thereof on the user terminal 60.

### Hardware Configuration Example

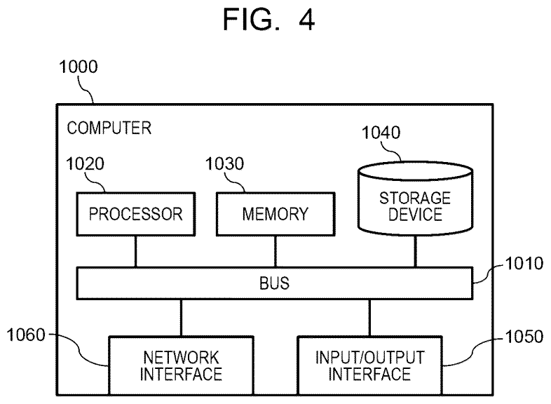

FIG. 4 is a block diagram illustrating a hardware configuration of a computer 1000 that implements the information processing apparatus 100 (user terminal 60). The wearable terminal 50 in FIG. 3 is also implemented by the computer 1000. Moreover, even in an example described in an example embodiment described later in which the information processing apparatus 100 is implemented by a combination with a server apparatus 70, the server apparatus 70 is implemented by the computer 1000. Moreover, a manager terminal 80 described in an example embodiment to be described later is also implemented by the information processing apparatus 100.

The computer 1000 includes a bus 1010, a processor 1020, a memory 1030, a storage device 1040, an input/output interface 1050, and a network interface 1060.

The bus 1010 is a data transmission path through which the processor 1020, the memory 1030, the storage device 1040, the input/output interface 1050, and the network interface 1060 transmit/receive data to/from one another. However, a method of mutually connecting the processor 1020 and the like is not limited to bus connection.

The processor 1020 is a processor implemented by a central processing unit (CPU), a graphics processing unit (GPU), or the like.

The memory 1030 is a main storage apparatus implemented by a random access memory (RAM) or the like.

The storage device 1040 is an auxiliary storage apparatus implemented by a hard disk drive (HDD), a solid state drive (SSD), a memory card, a read only memory (ROM), or the like. The storage device 1040 stores a program module that achieves each function (e.g., a computation unit 102, an estimation unit 104, and an update unit 106, and an acquisition unit 110, an exclusion unit 112, and an output processing unit 114, and the like described later) of the information processing apparatus 100. The processor 1020 reads each of the program modules onto the memory 1030, executes the read program module, and thereby achieves each function being relevant to the program module. Moreover, the storage device 1040 may also store each piece of data in a storage apparatus 120 described later.

The program module may be recorded on a medium. A medium that records the program module includes a non-transitory tangible medium usable by the computer 1000, and a program code readable by the computer 1000 (processor 1020) may be embedded in the medium.

The input/output interface 1050 is an interface for connecting the computer 1000 and various pieces of input/output equipment. The input/output interface 1050 also functions as a communication interface that performs near-field wireless communication such as Bluetooth (registered trademark) and near field communication (NFC).

The network interface 1060 is an interface for connecting the computer 1000 to a communication network. The communication network is, for example, a local area network (LAN) or a wide area network (WAN). A method of connecting the network interface 1060 to the communication network may be wireless connection or wired connection.

Then, the computer 1000 connects to necessary equipment (e.g., a display, a touch panel, display equipment, an operation button, a camera, a speaker, and a microphone of the user terminal 60, a display, a touch panel, display equipment, an operation button, a speaker, and a microphone of the wearable terminal 50, a keyboard, a mouse, a speaker, a microphone, and a printer of the server apparatus 70, and the like) via the input/output interface 1050 or the network interface 1060.

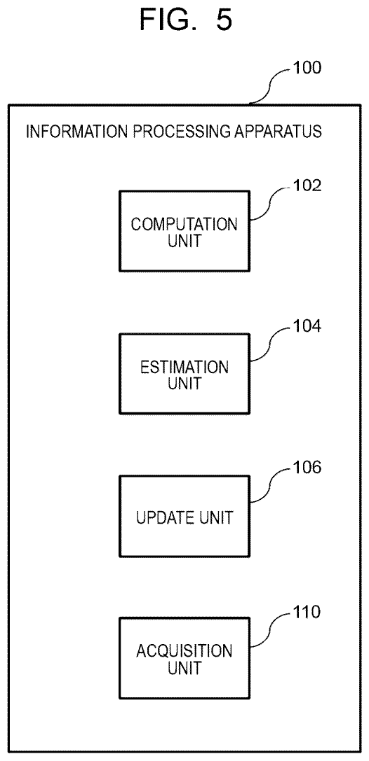

Each component of the information processing apparatus 100 according to each example embodiment in FIG. 1, and FIGS. 5, 16, 18, and 21 described later is achieved by any combination of hardware and software of the computer 1000 in FIG. 4. Then, it is appreciated by a person skilled in the art that there are various modified examples of methods and apparatuses for the achievement. A functional block diagram illustrating the information processing apparatus 100 according to each example embodiment illustrates not a configuration on a hardware basis but a block on a logical function basis.

### Functional Configuration Example

FIG. 5 is a functional block diagram illustrating a logical configuration example of the information processing apparatus 100 according to the example embodiment.

The information processing apparatus 100 includes the computation unit 102, the estimation unit 104, and the update unit 106 that are the same as those of the information processing apparatus 100 in FIG. 1, and further includes the acquisition unit 110.

The acquisition unit 110 acquires biometric information from the wearable terminal 50 worn by the user U (subject).

In the example embodiment, a pulse is described as an example of biometric information acquired by the acquisition unit 110. However, as described above, various types of biometric information can be utilized.

A timing of acquiring biometric information from the wearable terminal 50 is not particularly limited. According to a storage capacity on the wearable terminal 50, a certain amount of biometric information may be accumulated, and the biometric information for a predetermined period may be collectively transmitted. According to a communication state between the wearable terminal 50 and the user terminal 60, biometric information may be transmitted in a case where the communication situation is good, or may be transmitted immediately every time the biometric information is acquired or after elapse of a certain period of time.

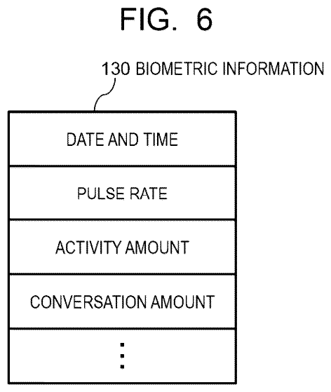

FIG. 6 is a diagram illustrating a data structure example of biometric information 130. The biometric information 130 stores time information, and biometric information such as a pulse rate, an activity amount, and a conversation amount, in association with each other. Biometric information 130 is stored in the memory 1030 or the storage device 1040 of the computer 1000 that implements the user terminal 60.

The computation unit 102 processes biometric information acquired by the acquisition unit 110, and thereby computes a score indicating a mental state of the subject U (subject). The score indicating the mental state includes a plurality of scores indicating a plurality of types of mental states, respectively. As one example, a score includes scores indicating an arousal level and a harmony level, respectively. For example, the arousal level is a value indicating an arousal degree indicating a state of drowsiness, and indicates that the arousal degree is in a state of being great as the score value is great, or indicates that a degree of drowsiness is in a state of being great as the score value is small. The harmony level is a value indicating an emotion such as comfort or discomfort, and indicates being in a comfortable state as the score value is great, or indicates an uncomfortable state as the score value is small.

The score may be, for example, a value indicated by a range of one of numerical values of 0 to 100, −100 to +100, 0 to 1, and −1 to +1, but is not limited thereto.

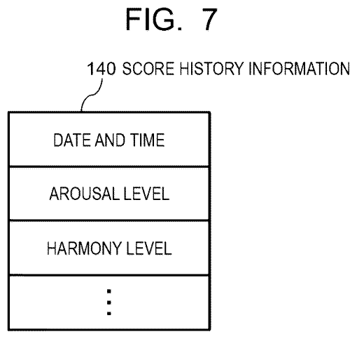

FIG. 7 is a diagram illustrating a data structure example of score history information 140. The score history information 140 stores time information, and a score computed by the computation unit 102, in association with each other. The score history information 140 is stored in the memory 1030 or the storage device 1040 of the computer 1000 that implements the user terminal 60.

The estimation unit 104 estimates an emotion of the user U (subject) by use of a criterion including a threshold value associated with the user U (subject) and a score indicating a mental state of the user U (subject). In the example embodiment, the estimation unit 104 estimates, from two scores indicating two types of mental states, which of four emotions an emotion of the user U is.

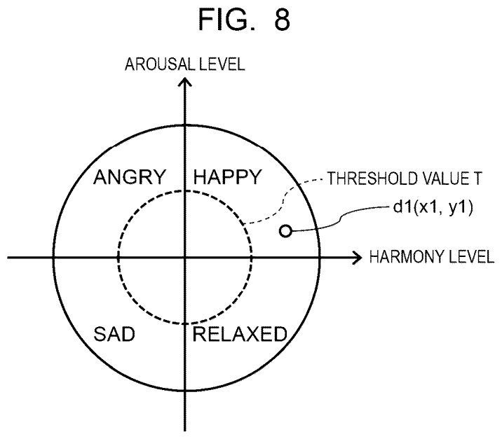

FIG. 8 is a diagram for describing an emotion estimation method using an arousal level and a harmony level.

In this example, the arousal level and the harmony level are indicated as coordinate axes, and the four regions acquired by quartering by the coordinate axis are relevant to four emotions of the user U. In this example, the four emotions are “ANGRY”, “HAPPY”, “SAD”, and “RELAXED”, and are set by four coordinate regions. Then, in a case where a score value of the user U falls within a range exceeding a threshold value, it is estimated that the user U is in an emotion of the range.

The estimation unit 104 plots values of scores indicating two types of mental states of the user U (subject) on a coordinate as values of two coordinate axes, respectively. The four regions acquired by quartering by the coordinate axis indicate four different emotional ranges of the user U (subject), respectively. Then, in a case where the plotted value exceeds the threshold value, the estimation unit 104 estimates that an emotion being relevant to the region where the plotted value exists is an emotion of the user U (subject).

Specifically, the estimation unit 104 plots a score indicating an arousal level and a score indicating a harmony level on a graph in FIG. 8. A threshold value T is previously set for at least one of the arousal level and the harmony level. The threshold value T is indicated by, for example, a value between 0 and 100 on a Y coordinate of the arousal level in FIG. 8. Then, a region beyond a circle with a radius based on the threshold value as a criterion and up to 100 on the Y axis is relevant to each emotion. That is to say, in a case where a plotted value of a score is included in each region exceeding the threshold value T, it is estimated that the user U (subject) has the emotion of the region. For example, FIG. 8 illustrates data d1 in which a score y1 of an arousal level and a score x1 of a harmony level at a time t1 are plotted. Since the data dl of the user U (subject) in this instance are included in the region indicated as “HAPPY” exceeding the threshold value T, it is estimated that the user U has a happy emotion.

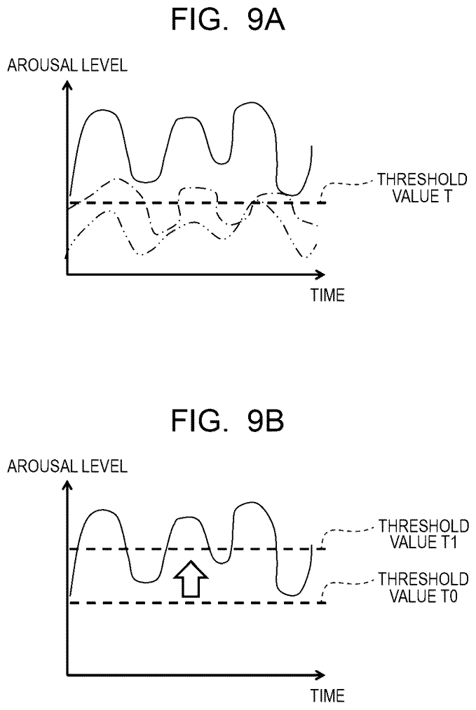

FIG. 9 is a diagram for describing an individual difference in biometric information and adjustment of a threshold value.

As illustrated in FIG. 9(a), a time change in an average arousal level is illustrated by a dashed-dotted line, and the average threshold value T is illustrated by a broken line. On the other hand, as illustrated in FIG. 9(a), there is a person who shows a change in an arousal level as illustrated by a solid line, and there is also a person who shows a change in an arousal level as illustrated by a two-dot chain line. In this way, there is an individual difference in a score indicating a mental state of a subject.

For a subject indicated by the solid line, in a case where an emotion is estimated by use of the threshold value T, a score of an arousal level always exceeds the threshold value T, and, therefore, there is a risk that, for example, the subject is estimated to be always angry.

Accordingly, the update unit 106 updates the threshold value T by use of a history of the score.

That is to say, as illustrated in FIG. 9(b), regarding a subject with a score history of an arousal level indicated by the solid line, a threshold value T0 is changed to a threshold value T1. That is to say, the threshold value T1 is set to be higher than the threshold value T0. Thereby, since there is no longer a case as described above where a score of an arousal level always exceeds a threshold value, this allows an emotion of a subject to be accurately estimated.

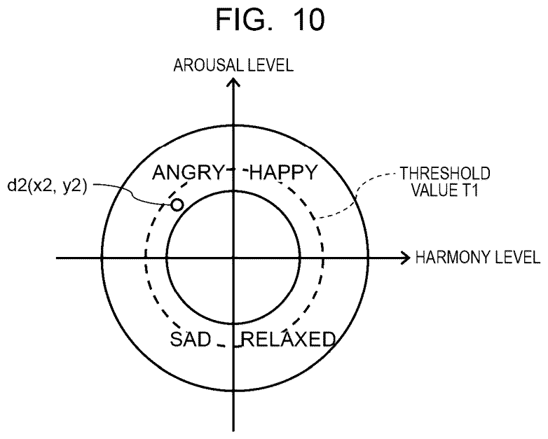

FIG. 10 is a diagram for describing an emotion analysis using the threshold value T1 updated by the update unit 106.

For example, data d2 of the user U at a time t2 exist inside the threshold value T1, and therefore exist outside the region of “ANGRY”. Thus, it is estimated that an emotion of the subject in this instance is not “ANGRY”.

The update unit 106 sets a threshold value for the user U (subject) by use of a maximum value and a minimum value of a score of the user U (subject).

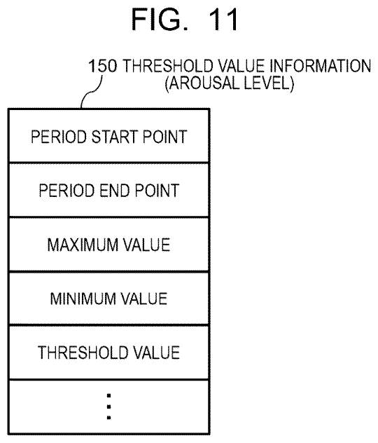

FIG. 11 is a diagram illustrating a data structure example of threshold value information 150. In this example, the threshold value information 150 includes information for updating a threshold value of an arousal level. The threshold value information 150 stores, in association with each other, time information of a period start point and a period end point indicating a period of the score history information 140 used for computing the threshold value, a maximum value and a minimum value of a score of an arousal level in the period, and a threshold value updated by an average value computed from the maximum value and the minimum value.

However, the maximum value and the minimum value of a score of an arousal level may not be stored. The threshold value information 150 is stored in the memory 1030 or the storage device 1040 of the computer 1000 that implements the user terminal 60.

### Operation Example

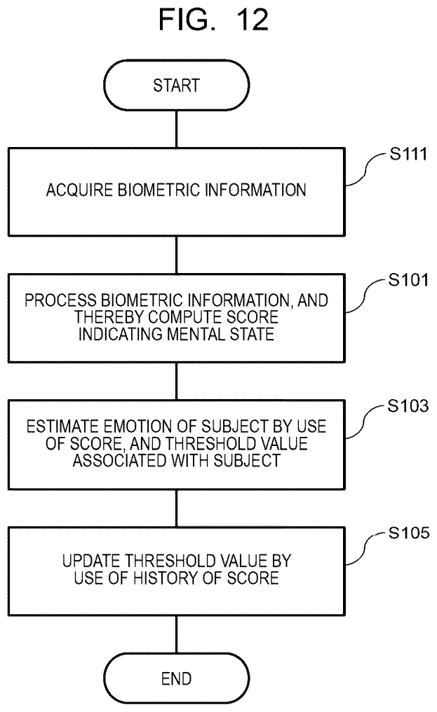

FIG. 12 is a flowchart illustrating an operation example of the information processing apparatus 100 according to the example embodiment.

The flow in FIG. 12 includes steps S101 to S105 being the same as those in the flow in FIG. 2, and further includes step S111. However, each step in the flowchart is independently operable, and, as described above, a timing of being executed may be different for each step.

First, the acquisition unit 110 acquires biometric information from the wearable terminal 50 worn by the user U (step S111). The acquired biometric information is stored in the memory 1030 or the storage device 1040 of the user terminal 60 as the biometric information 130 in association with time information. The time information is preferably a time when biometric information is measured by the wearable terminal 50, but may be a time when the biometric information is received from the wearable terminal 50, or may be a time when the biometric information is stored as the biometric information 130.

The computation unit 102 processes biometric information of the user U, and thereby computes a score indicating a mental state of the user U (step S101). The computed score is stored as the score history information 140 in the memory 1030 or the storage device 1040 of the user terminal 60.

The estimation unit 104 estimates an emotion of the user U by use of the score and a criterion including a threshold value associated with the user U (step S103). An estimated result is stored as score history information 140 in the memory 1030 or the storage device 1040 of the user terminal 60.

The update unit 106 updates the threshold value by use of a history of the score (step S105). For example, by use of a score history for one week, the update unit 106 extracts a maximum value and a minimum value of a score, computes an average value, and sets a threshold value. The computed threshold value is stored as the threshold value information 150 in the memory 1030 or the storage device 1040 of the user terminal 60. Then, the updated threshold value is used by the estimation unit 104 as a threshold value for the user U for a next week.

However, in a case where a score value includes a negative value (e.g., on a drowsiness side of an arousal level), the update unit 106 computes an average value of at least one of a score value of a positive value (arousal level) and a score value of a negative value (drowsiness level), and determines the average value as a threshold value.

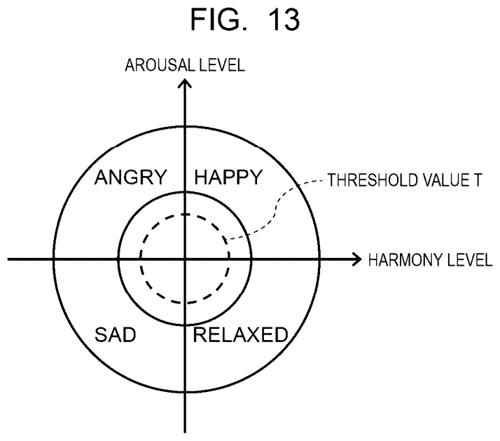

Note that, in a case where the threshold value T updated by the update unit 106 is lower than a previous one, a region of each emotion becomes wider as illustrated in FIG. 13.

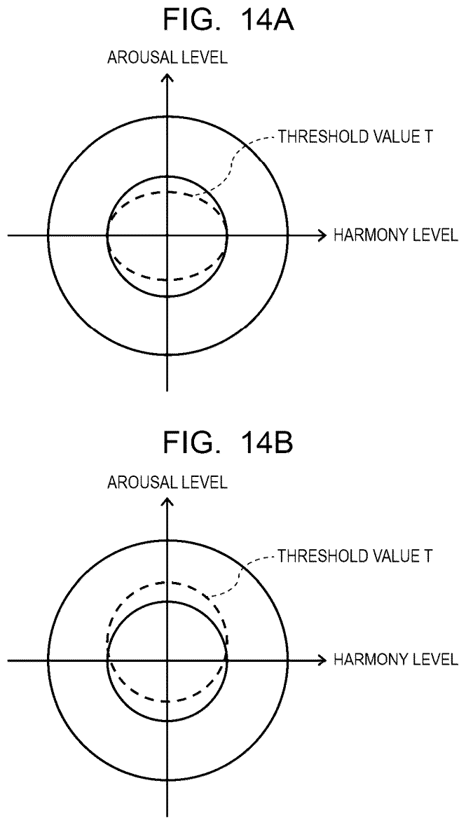

For example, the update unit 106 may update a threshold value for each type of score. For example, as illustrated in FIG. 14(a), only the threshold value T of an arousal level may be updated. Further, the update unit 106 analyzes a tendency of a change in score, and, in a case of the user U whose score value exists upward as a whole as a solid line in FIG. 9(b), the threshold value T of an arousal level may be changed in such a way that the threshold value T is shifted upward as a whole as illustrated in FIG. 14(b). For example, the update unit 106 may hold a minimum value and a maximum value of a score to be a standard, and determine to change method of the threshold value by comparing standard values of the minimum value and the maximum value of the user U. That is to say, in a case where the minimum value and the maximum value of the user U both indicate values lower than the standard value, the threshold value of the user U may be changed in such a way that the threshold value of the user U is shifted lower than the standard threshold value.

As described above, according to the present example embodiment, the information processing apparatus 100 includes the computation unit 102, the estimation unit 104, the update unit 106, and the acquisition unit 110. The acquisition unit 110 acquires biometric information from the wearable terminal 50 worn by the user U. The computation unit 102 processes biometric information of the user U, and thereby computes a score indicating a mental state of the user U. The estimation unit 104 estimates an emotion of the user U by use of the score and a criterion including a threshold value associated with the user U. The update unit 106 updates the threshold value by use of a history of the score.

In this way, since the information processing apparatus 100 according to the example embodiment can update a threshold value used for an emotion analysis by use of a history of a score indicating a mental state of the user U, and use the updated threshold value for the next emotion analysis, an individual difference in biometric information can be considered, and accuracy can be improved in the emotion analysis using biometric information.

### Second Example Embodiment

### System Outline

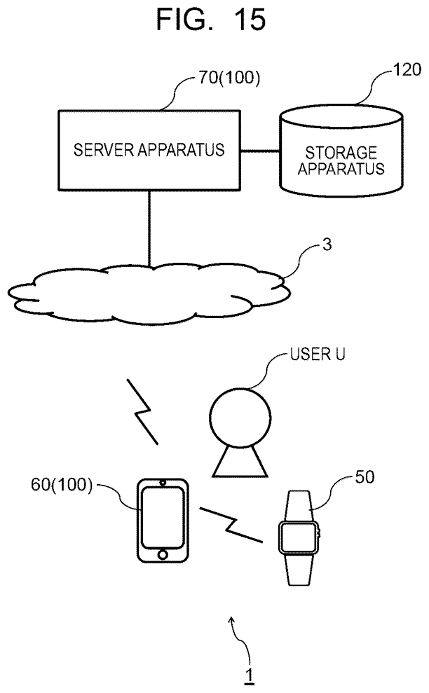

FIG. 15 is a diagram conceptually illustrating a system configuration of an information processing system 1 according to the example embodiment. The information processing system 1 further includes a server apparatus 70, in addition to the information processing system 1 according to the example embodiment illustrated in FIG. 3.

In the example embodiments described above, an information processing apparatus 100 is implemented by a user terminal 60, but, in the present example embodiment, the information processing apparatus 100 is implemented by a combination of the user terminal 60 and the server apparatus 70. Moreover, the information processing apparatus 100 according to the present example embodiment is the same as the example embodiment described above except for including a configuration that excludes, from a score history used for updating a threshold value, the score for a period satisfying a specific condition. Note that, the configuration according to the present example embodiment may be combined with at least one of the configurations according to other example embodiments to an extent that no contradiction occurs.

The server apparatus 70 is a server computer implemented by a computer 1000 in FIG. 4 described above. Moreover, the server apparatus 70 may include a web server. The user terminal 60 is connected to the server apparatus 70 via a communication network 3. The communication network 3 may be a network such as the Internet, or may be a network within a company. The server apparatus 70 includes a storage apparatus 120. The storage apparatus 120 may be provided inside the server apparatus 70, or may be provided outside. That is to say, the storage apparatus 120 may be hardware integrated with the server apparatus 70, or may be hardware separate from the server apparatus 70.

In the example embodiment described above, the information processing apparatus 100 is implemented only by the user terminal 60, but in the present example embodiment, the information processing apparatus 100 is implemented by a combination of the server apparatus 70 and the user terminal 60. Moreover, it is assumed that a user U can utilize a service provided by the present information processing system 1 by accessing the server apparatus 70 by use of the user terminal 60. Thus, the user U previously performs user registration, and sets account information. In a case of utilizing a service of the information processing system 1, the user U logs into the information processing system 1 by use of the account information.

The application described above is installed on the user terminal 60, and the user U can utilize a service provided by the information processing system 1, by starting the application, and logging into the information processing system 1. Alternatively, the user U may be able to utilize a service provided by the information processing system 1, by starting a predetermined browser on the user terminal 60, accessing a website for utilizing the service provided by the information processing system 1, and logging in by use of the account information.

### Functional Configuration Example

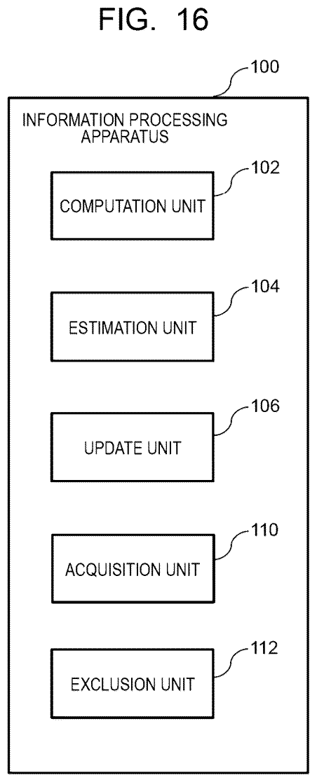

FIG. 16 is a diagram illustrating an outline of the information processing apparatus 100 according to the example embodiment. The information processing apparatus 100 further includes an exclusion unit 112, in addition to the configuration of the information processing apparatus 100 in FIG. 5.

The exclusion unit 112 excludes a score for a period satisfying a specific condition in a history of a score.

The specific condition is, for example, a period specified by the user U. In addition to a period specified by the user U, the specific condition may be a numerical value range of a score specified by the user U. The exclusion unit 112 displays, on an operation screen of an application, a user interface (UI) that accepts specification of a period desired to be excluded in the score history information 140, and accepts the specification of the user U. In the present example embodiment, for example, the server apparatus 70 can execute at least part of the functions of a computation unit 102, an estimation unit 104, an update unit 106, an acquisition unit 110, and the exclusion unit 112. Biometric information is information that can also be said to be individual information of the user U. Thus, the present example embodiment allows transmission to the server apparatus 70 in such a way that biometric information for a period that the user U does not desire to transmit to the server apparatus 70 is excluded. Moreover, in addition to a reason that the user U “does not desire to transmit”, biometric information in a case of being in a mental state significantly different from a normal one may be excluded.

Function allocation of the information processing apparatus 100 to the user terminal 60 and the server apparatus 70 is illustrated below.
(a1) The user terminal 60 achieves a function of the acquisition unit 110 of acquiring biometric information from a wearable terminal 50, and a function of the exclusion unit 112, and the server apparatus 70 achieves a function of the acquisition unit 110 of acquiring biometric information from the user terminal 60, and functions of the computation unit 102, the estimation unit 104, and the update unit 106.
(a2) The user terminal 60 achieves a function of the acquisition unit 110 of acquiring biometric information from the wearable terminal 50, and the server apparatus 70 achieves a function of the acquisition unit 110 of acquiring biometric information from the user terminal 60, and functions of the exclusion unit 112, the computation unit 102, the estimation unit 104, and the update unit 106.
(a3) The user terminal 60 achieves functions of the acquisition unit 110, the exclusion unit 112, the computation unit 102, the estimation unit 104, and the update unit 106, and a function of transmitting an estimation result the estimation unit 104 of to the server apparatus 70, and the apparatus 70 achieves a function of recording and presenting an estimation result received from the user terminal 60.
(a4) The user terminal 60 achieves functions of the acquisition unit 110, the exclusion unit 112, the computation unit 102, and the estimation unit 104, and a function of transmitting a score computed by the computation unit 102 and an estimation result of the estimation unit 104 to the server apparatus 70, and the server apparatus 70 achieves a function of recording and presenting an estimation result received from the user terminal 60, and a function of the update unit 106 by use of a score received from the user terminal 60.

Moreover, the acquisition unit 110 achieved by the server apparatus 70 may acquire biometric information from the user terminal 60 of the user U as described above, or may read and acquire biometric information transitorily stored in an external storage apparatus (or a storage medium) from the user terminal 60 of the user U, from another storage apparatus. The computation unit 102 computes a score by use of the biometric information acquired by the acquisition unit 110.

For example, the biometric information acquired by the acquisition unit 110 from the wearable terminal 50 is stored in a memory 1030 or a storage device 1040 of the user terminal 60. In a case where score history information 140 for a predetermined period is accumulated, the exclusion unit 112 displays, on a display of the user terminal 60 at a predetermined timing, a screen including a UI that accepts specification of at least either a period of permitting the user U to transmit to the server apparatus 70 or a period to be excluded from information to be transmitted. Alternatively, the exclusion unit 112 may cause the user terminal 60 to display a screen including a message indicating, to the user U, a period to be a transmission target and inquiring whether biometric information for the period may be transmitted to the server apparatus 70, and a UI that accepts a response of approval or rejection.

In a case where specification of a period for which the user U permits transmission or a period to be excluded is accepted, the exclusion unit 112 transmits, to the server apparatus 70, biometric information for the permitted period or biometric information excluding the period specified to be excluded. Alternatively, upon accepting a response agreeing to a period to be a transmission target, the exclusion unit 112 transmits, to the server apparatus 70, biometric information for the period of the transmission target. Upon accepting a response rejecting transmission for a period to be a transmission target, the exclusion unit 112 may cause the user terminal 60 to display a screen including a UI that accepts specification of at least either a period of permitting the server apparatus 70 to transmit or a period to be excluded from information to be transmitted, similarly accept specification of a period of permitting transmission or a period to be excluded, and transmit biometric information to the server apparatus 70 according to the specification.

Alternatively, the exclusion unit 112 may previously accept, on a setting screen, specification of a period to be determined as a transmission target or a period to be excluded from a transmission target. For example, the exclusion unit 112 may accept specification of a day of a week, a time period, and the like to be determined as a transmission target, or specification of a day of a week to be excluded from a transmission target. The exclusion unit 112 transmits biometric information to the server apparatus 70 according to a previously specified period.

The update unit 106 updates a threshold value by use of a score excluding a score for a period satisfying a specific condition from among a history by the exclusion unit 112.

In a case where the update unit 106 is achieved by the server apparatus 70, since biometric information from which a period satisfying a specific condition is excluded by the exclusion unit 112 is transmitted to the server apparatus 70, the update unit 106 updates a threshold value by use of the received biometric information. On the other hand, in a case where the update unit 106 is achieved by the user terminal 60, the update unit 106 updates a threshold value by use of biometric information from which a period satisfying a specific condition is excluded by the exclusion unit 112.

The estimation unit 104 estimates an emotion of the user U (subject) by use of a score from which a score for a period satisfying a specific condition is excluded in a history by the exclusion unit 112.

In a case where the estimation unit 104 is achieved by the server apparatus 70, since biometric information from which a period satisfying a specific condition is excluded by the exclusion unit 112 is transmitted to the server apparatus 70, the estimation unit 104 updates a threshold value by use of the received biometric information. On the other hand, in a case where the estimation unit 104 is achieved by the user terminal 60, the estimation unit 104 updates a threshold value by use of biometric information from which a period satisfying a specific condition is excluded by the exclusion unit 112.

The computation unit 102 may compute an average value of a score for each predetermined period, and the exclusion unit 112 may determine a period to be excluded, by use of an average value satisfying a specific condition.

A period is, for example, one week or the like. The computation unit 102 computes an average value of a score of the user U for each week, and the exclusion unit 112 excludes the score for the one week in a case where a difference in average value from another week is equal to or more than a predetermined value. The predetermined period may be in a one-day unit, and is not particularly limited. By excluding a score for a period in which a difference between a computed average value and that in another period is equal to or more than a predetermined value, update processing of a threshold value using the score in the period can be prevented from being performed in a case where a score greatly fluctuates temporarily in contrast to a normal one but not due to an individual difference. That is to say, a threshold value can be prevented from receiving an influence of a temporary fluctuation in score different from a normal one.

The exclusion unit 112 may acquire schedule information of the user U (subject). Then, the exclusion unit 112 determines, by use of the schedule information, a period to be excluded from a processing target.

For example, schedule information of user U includes information indicating a break time of the user U, and the exclusion unit 112 determines the break time of the user U as a period to be excluded from the processing target. Moreover, a setting screen including a UI that accepts specification of which period to exclude in the schedule information may be displayed on the user terminal 60, and specification of the user U may be accepted. For example, in a case where a plan for a presentation to a customer is in a schedule, the user U knows that the user U becomes nervous, and, therefore, the user U may be able to specify that biometric information for a time of the plan is excluded. Thereby, since a score in a case where a mental state of the user U is greatly different from a normal one can be excluded, update processing of a threshold value using the score for the period can be prevented from being performed. That is to say, a threshold value can be prevented from receiving an influence of a temporary fluctuation in score different from a normal one.

### Operation Example

An operation of the information processing apparatus 100 according to the example embodiment is described by use of FIG. 12. Herein, a function allocation example of (a1) is described.

First, the acquisition unit 110 acquires biometric information from the wearable terminal 50 worn by the user U (step S111). The acquired biometric information is stored in the memory 1030 or the storage device 1040 of the user terminal 60 as biometric information 130 in association with time information. Then, the acquisition unit 110 transmits biometric information accumulated for a predetermined period, from the user terminal 60 to the server apparatus 70 at a predetermined timing. Note that, before the acquisition unit 110 transmits the biometric information, the exclusion unit 112 excludes the score for a period satisfying a specific condition.

In the server apparatus 70, the acquisition unit 110 causes the storage apparatus 120 to store, in association with identification information of the user U, the biometric information 130 of the user U received from the user terminal 60. Then, in the server apparatus 70, the computation unit 102 processes biometric information of the user U, and thereby computes a score indicating a mental state of the user U (step S101). The computed score is stored in the storage apparatus 120 as the score history information 140.

Then, in the server apparatus 70, the estimation unit 104 estimates an emotion of the user U by use of the score and a criterion including a threshold value associated with the user U (step S103). An estimated result is stored in the storage apparatus 120 as the score history information 140.

Then, the update unit 106 updates the threshold value by use of a history of the score (step S105). For example, by use of a score history for one week, the update unit 106 extracts a maximum value and a minimum value of a score, computes an average value, and sets a threshold value. The computed threshold value is stored in the storage apparatus 120 as threshold value information 150.

The threshold value updated in this way is used by the estimation unit 104 as a threshold value for the user U for a next week.

As described above, according to the present example embodiment, the exclusion unit 112 is provided. The exclusion unit 112 excludes the score for a period satisfying a specific condition in a history of a score. Then, the update unit 106 updates a threshold value by use of a score from which a score for a period is excluded in a history by the exclusion unit 112. Further, the estimation unit 104 estimates an emotion of the user U by use of the score from which the score for the period is excluded in the history by the exclusion unit 112.

In this way, the information processing apparatus 100 according to the present example embodiment provides a similar effect to that according to the example embodiment described above, further, the user U can specify biometric information and the like that the user U does not desire to be utilized for an emotion analysis, and the estimation unit 104 can perform the emotion analysis in such a way that biometric information for the specified period is excluded by the exclusion unit 112. Furthermore, since the update unit 106 can update a threshold value by use of a score from which a period satisfying a specific condition is excluded, accuracy of the threshold value can be improved by performing update of the threshold value in such a way that a score in a case of a state different from a normal mental state is excluded.

### Third Example Embodiment

### System Outline

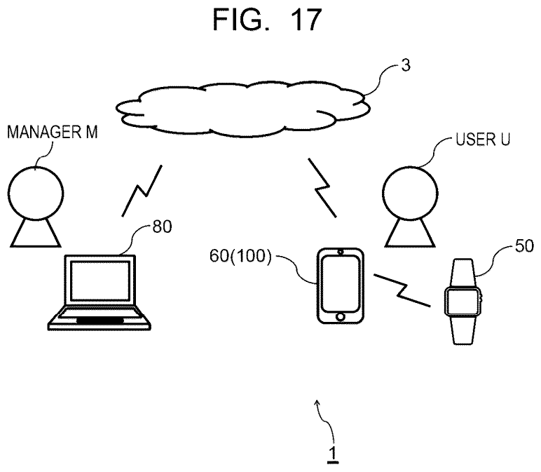

FIG. 17 is a diagram conceptually illustrating a system configuration of an information processing system 1 according to the example embodiment. In addition to the information processing system 1 according to the example embodiment illustrated in FIG. 3, the information processing system 1 further includes a manager terminal 80. The manager terminal 80 is, for example, a terminal utilized by a manager M who manages a user U in a company where the user U works, and is a computer 1000 such as a personal computer, a tablet terminal, or a smartphone. The manager terminal 80 can communicate with a user terminal 60 via a communication network 3.

The present example embodiment is similar to the first example embodiment except for including a configuration that outputs such a fact to an external terminal (manager terminal 80) in a case where an estimation result of an emotion satisfies a criterion. However, the configuration according to the present example embodiment may be combined with at least one of the configurations according to example embodiments other than the first example embodiment to an extent that no contradiction occurs.

### Functional Configuration Example

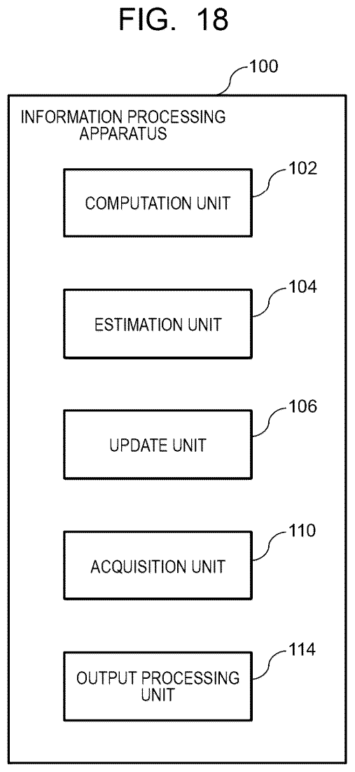

FIG. 18 is a functional block diagram illustrating a functional configuration example of an information processing apparatus 100 according to the example embodiment. In addition to components in FIG. 5, the information processing apparatus 100 further includes an output processing unit 114.

In a case where an estimation result of an emotion satisfies a criterion, the output processing unit 114 causes the manager terminal 80 (external terminal) to output such a fact.

The criterion is, for example, that an estimation result of an emotion indicates a state where the user U (subject) is under severe stress, that an estimation result of an emotion indicates that anxiety of the user U (subject) continues for equal to or more than a certain period of time, or the like.

That is to say, the criterion is that an estimation result of an emotion indicates that a situation has arisen where the manager M needs to know and appropriately deal with a mental state of the user U. A content to be output includes, for example, information with which the user U to be a notification target is identifiable, for example, a department to which the user U belongs, a name, and the like, and a content of an estimation result of an emotion satisfying a criterion. User information including the department to which the user U belongs and the name is previously stored in the memory 1030 or the storage device 1040 of the user terminal 60 (in the case of the server apparatus 70, the storage apparatus 120) in association with the identification information of the user U. In the case of the server apparatus 70, the output processing unit 114 can acquire and include, in an output content, the department to which the user U belongs and the name associated with the identification information of the subject (user U) who needs notification.

The output processing unit 114 transmits, to the manager terminal 80, a message notifying that the estimation result of an emotion of the user U satisfies the criterion, for example, by e-mail, a message of short message service (SMS), or the like. Destination information of a transmission destination is previously stored in the storage device 1040 of the user terminal 60.

In an example in which the output processing unit 114 is achieved by the server apparatus 70 described in an example embodiment described later, the manager M previously performs user registration, and sets account information, for example, similarly to the user terminal 60. In a case of utilizing a service of the information processing system 1, the user U logs into the information processing system 1 by use of account information.

The manager M can utilize the service provided by the information processing system 1 by installing the application described above on the manager terminal 80, starting the application, and logging into the information processing system 1. Alternatively, the manager M may be able to utilize the service provided by the information processing system 1 by starting a predetermined browser on the manager terminal 80, accessing a website for utilizing the service provided by the information processing system 1, and logging in by use of account information.

Then, in the server apparatus 70, the output processing unit 114 notifies, on the manager terminal 80, the manager M that the estimation result of an emotion of the user U (subject) satisfies the criterion, on a notification screen on the application or a website.

Moreover, the user U may be able to specify permission or non-permission of a notification function by the output processing unit 114 to the manager terminal 80. The output processing unit 114 can accept specification by a specification screen including a UI that accepts a selection of permission or non-permission. According to the specification of the user U, the output processing unit 114 decides whether to notify the manager terminal 80. Alternatively, before notifying the manager terminal 80, the output processing unit 114 may cause the user terminal 60 to display a screen including a UI that accepts a notification content and specification of permission or non-permission of a notification, and decide approval or disapproval of notification to the manager terminal 80 according to specification of the user U.

### Operation Example

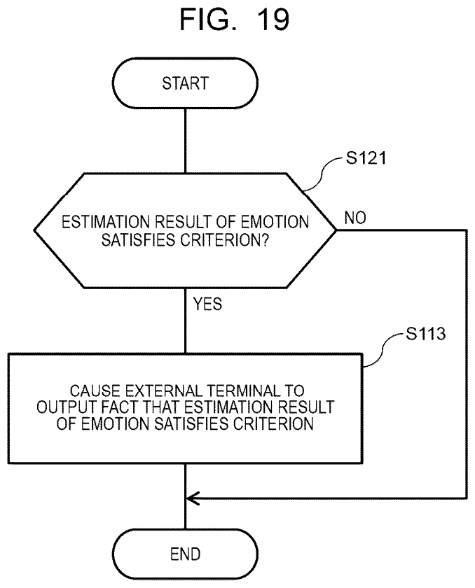

FIG. 19 is a flowchart illustrating an operation example of the information processing apparatus 100 according to the example embodiment.

The flow may be executed independently of the flow in FIG. 2 or 12, and may be executed at any time or periodically at a predetermined timing. For example, the flow may be executed every week, every month, or the like. Alternatively, the manager M may be able to specify execution, or the flow may be executed in a case where specification of the manager M is accepted.

The output processing unit 114 decides whether an estimation result of an emotion of the user U estimated by the estimation unit 104 satisfies a criterion (step S121). Then, in a case where the estimation result of an emotion satisfies the criterion (YES in step S121), the output processing unit 114 causes the manager terminal 80 (external terminal) to output a fact that the estimation result of an emotion of the user U satisfies the criterion (step S113). The output processing unit 114 acquires and includes, in an output content, a department to which the user U belongs, a name, and the like, and transmits a message to the manager terminal 80 of the manager M. In a case where the estimation result of an emotion does not satisfy the criterion (NO in step S121), step S113 is bypassed, and the present processing ends.

In a case where the server apparatus 70 achieves a function of the output processing unit 114, the output processing unit 114 acquires an estimation result of an emotion for each of the users U, decides whether the estimation result of an emotion satisfies the criterion for each of the users U, and, in a case where the criterion is satisfied, acquires and includes, in an output content, a department to which the user U belongs, a name, and the like associated with identification information of the user U. Then, the output processing unit 114 outputs a notification screen to an application of the manager terminal 80 of the manager M or a web page for the manager M.

As described above, according to the present example embodiment, the output processing unit 114 is provided. In a case where an estimation result of an emotion satisfies a criterion, the output processing unit 114 causes the manager terminal 80 (external terminal) to output such a fact.

In this way, since the information processing apparatus 100 according to the present example embodiment provides a similar effect to that according to the example embodiment described above, and, further, can cause, in a case where an estimation result of an emotion of the user U (subject) satisfies the criterion, the manager terminal 80 of the manager M to output such a fact, the manager M can quickly know and appropriately deal with a mental state of the user U in case of necessity. The user U can cause the manager M to know a mental state and take an appropriate dealing, and therefore becomes able to work with relief.

### Fourth Example Embodiment

### System Outline

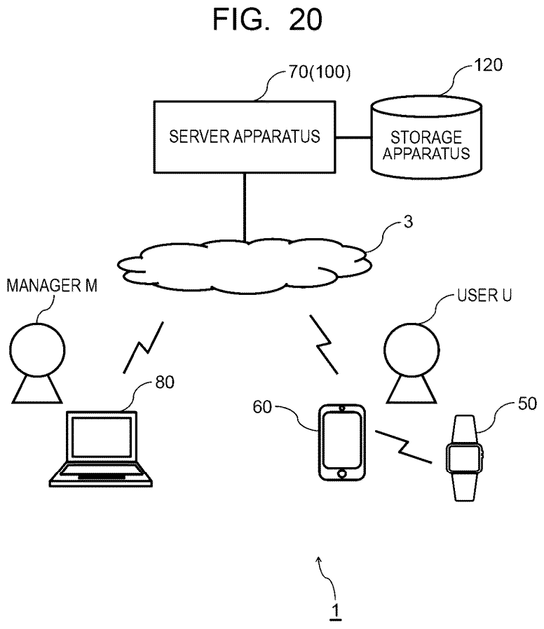

FIG. 20 is a diagram conceptually illustrating a system configuration of an information processing system 1 according to an example embodiment. The information processing system 1 includes a server apparatus 70 and a manager terminal 80 in addition to the information processing system 1 according to the example embodiment in FIG. 3. In other words, in addition to the information processing system 1 according to the example embodiment illustrated in FIG. 15, the manager terminal 80 is provided.

### Functional Configuration Example

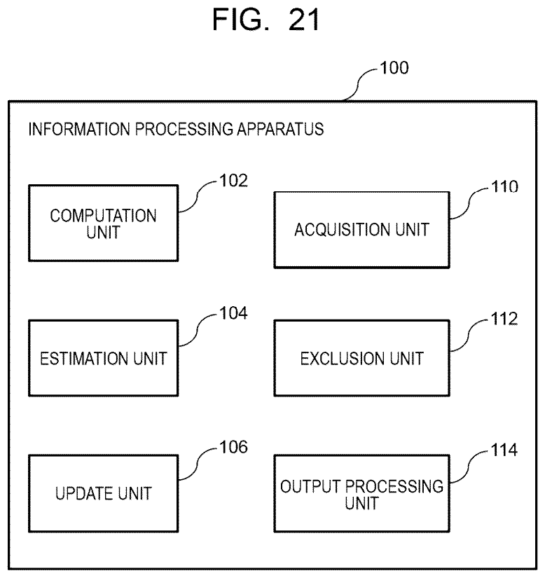

FIG. 21 is a diagram illustrating an outline of the information processing apparatus 100 according to the example embodiment. In the present example embodiment, the information processing apparatus 100 is implemented by a combination of a user terminal 60 and the server apparatus 70. Moreover, the information processing apparatus 100 includes all units described in the example embodiments described above. That is to say, the information processing apparatus 100 includes a computation unit 102, an estimation unit 104, an update unit 106, an acquisition unit 110, an exclusion unit 112, and an output processing unit 114.

Function allocation of the information processing apparatus 100 to the user terminal 60 and the server apparatus 70 is illustrated below.
(b1) The user terminal 60 achieves a function of the acquisition unit 110 of acquiring biometric information from the wearable terminal 50, and a function of the exclusion unit 112, and the server apparatus 70 achieves a function of the acquisition unit 110 of acquiring biometric information from the user terminal 60, and functions of the computation unit 102, the estimation unit 104, the update unit 106, and the output processing unit 114.
(b2) The user terminal 60 achieves a function of the acquisition unit 110 of acquiring biometric information from the wearable terminal 50, and the server apparatus 70 achieves a function of the acquisition unit 110 of acquiring biometric information from the user terminal 60, and functions of the exclusion unit 112, the computation unit 102, the estimation unit 104, the update unit 106, and the output processing unit 114.
(b3) The user terminal 60 achieves functions of the acquisition unit 110, the exclusion unit 112, the computation unit 102, the estimation unit 104, the update unit 106, and the output processing unit 114, and a function of transmitting an estimation result of the estimation unit 104 to the server apparatus 70, and the server apparatus 70 achieves a function of recording and presenting an estimation result received from the user terminal 60.
(b4) The user terminal 60 achieves functions of the acquisition unit 110, the exclusion unit 112, the computation unit 102, the estimation unit 104, and the output processing unit 114, and a function of transmitting a score computed by the computation unit 102 and an estimation result of the estimation unit 104 to the server apparatus 70, and the server apparatus 70 achieves a function of recording and presenting an estimation result received from the user terminal 60, and a function of the update unit 106 by use of a score received from the user terminal 60.

### Operation Example

An operation of the information processing apparatus 100 according to the example embodiment is described by use of FIGS. 12 and 19. Herein, a function allocation example of (b1) is described.

First, the acquisition unit 110 acquires biometric information from the wearable terminal 50 worn by a user U (step S111 in FIG. 12). The acquired biometric information is stored in a memory 1030 or a storage device 1040 of the user terminal 60 as biometric information 130 in association with time information. Then, the acquisition unit 110 transmits the biometric information accumulated for a predetermined period, from the user terminal 60 to the server apparatus 70 at a predetermined timing. Note that, before the acquisition unit 110 transmits the biometric information, the exclusion unit 112 excludes the score for a period satisfying a specific condition.

In the server apparatus 70, the acquisition unit 110 causes the storage apparatus 120 to store the biometric information 130 of the user U received from the user terminal 60 in association with identification information of the user U. Then, in the server apparatus 70, the computation unit 102 processes biometric information of the user U, and thereby computes a score indicating a mental state of the user U (step S101 in FIG. 12). The computed score is stored in the storage apparatus 120 as score history information 140.

Then, in the server apparatus 70, the estimation unit 104 estimates an emotion of the user U by use of the score and a criterion including a threshold value associated with the user U (step S103 in FIG. 12). An estimated result is stored in the storage apparatus 120 as the score history information 140.

Then, the update unit 106 updates the threshold value by use of a history of the score (step S105 in FIG. 12). For example, by use of a score history for one week, the update unit 106 extracts a maximum value and a minimum value of a score, computes an average value, and sets a threshold value. The computed threshold value is stored in the storage apparatus 120 as threshold value information 150.

The threshold value updated in this way is used by the estimation unit 104 as a threshold value for user U for a next week.

On the other hand, the output processing unit 114 decides whether an estimation result of an emotion for each of the users U estimated by the estimation unit 104 satisfies a criterion (step S121 in FIG. 19). Then, regarding the user U for whom an estimation result of an emotion satisfies the criterion (YES in step S121 in FIG. 19), the output processing unit 114 causes the manager terminal 80 (external terminal) to output a fact that the estimation result of an emotion of the user U satisfies the criterion (step S113 in FIG. 19). The output processing unit 114 acquires and includes, in an output content, a department to which the user U belongs, a name, and the like, and transmits a message to the manager terminal 80 of a manager M. Alternatively, a notification screen including an output content is displayed on an application of the manager terminal 80 or a web page for the manager M. Regarding the user U for whom an estimation result of an emotion does not satisfy the criterion (NO in step S121 in FIG. 19), step S113 in FIG. 19 is bypassed, and the processing in FIG. 19 ends.

As described above, according to the present example embodiment, the information processing system 1 includes the wearable terminal 50, the user terminal 60, the server apparatus 70, and the manager terminal 80. The information processing apparatus 100 includes the computation unit 102, the estimation unit 104, the update unit 106, the acquisition unit 110, the exclusion unit 112, and the output processing unit 114. Then, each function of each unit of the information processing apparatus 100 is implemented by the user terminal 60 and the server apparatus 70 by function allocation.

In this way, the information processing apparatus 100 according to the present example embodiment provides a similar effect to that according to the example embodiment described above. That is to say, since a threshold value used for an emotion analysis can be updated by use of a history of a score indicating a mental state of user U, and an updated threshold value can be used for the next emotion analysis, an individual difference in biometric information can be considered, and accuracy can be improved in the emotion analysis using biometric information. Moreover, the user U can specify biometric information and the like that the user U does not desire to be utilized for the emotion analysis. Alternatively, accuracy of a threshold value can be improved by performing update of the threshold value in such a way that a score in a case of a state different from a normal mental state is excluded. Moreover, the manager M can quickly know and appropriately deal with a mental state of the user U in case of necessity. The user U can cause the manager M to know a mental state and take an appropriate dealing, and therefore becomes able to work with relief.

The example embodiments according to the present invention have been described above with reference to the drawings, but are exemplifications of the present invention, and various configurations other than those described above can also be adopted.

For example, in the example embodiment described above, the update unit 106 updates a threshold value by use of a history of a score of the user U. As another example embodiment, in a case where a threshold value is updated by the update unit 106, an update history of the threshold value may be recorded, the output processing unit 114 may monitor a tendency of a change in threshold value, and, in a case where update of the threshold value is continued, the manager terminal 80 may be caused to output such a fact. In a case of this configuration, it is possible to cope with, for example, such a case that the user U gradually becomes depressed.

Moreover, although a plurality of processes (pieces of processing) are described in order in a plurality of flowcharts used in the above description, an execution order of processes executed in each example embodiment is not limited to the described order. In each example embodiment, an order of illustrated processes can be changed to an extent that causes no problem in terms of content. Moreover, the example embodiments described above can be combined to an extent that content does not contradict.

While the invention of the present application has been described above with reference to the example embodiments, the invention of the present application is not limited to the example embodiments described above. Various changes that can be understood by a person skilled in the art can be made to the configuration and details of the invention of the present application within the scope of the invention of the present application.

Note that, in a case where information relating to a user is acquired and utilized in the present invention, this is performed legally.

Some or all of the above-described example embodiments can also be described as, but are not limited to, the following supplementary notes.
1. An information processing apparatus including:
   a computation unit that processes biometric information of a subject, and thereby computes a score indicating a mental state of the subject;
   an estimation unit that estimates an emotion of the subject by use of a criterion including a threshold value associated with the subject, and the score; and an update unit that updates the threshold value by use of a history of the score.
2. The information processing apparatus according to 1, wherein
   the update unit sets the threshold value of the subject by use of a maximum value and a minimum value of the score of the subject.
3. The information processing apparatus according to 1 or 2, further including
   an exclusion unit that excludes the score for a period satisfying a specific condition in the history of the score.
4. The information processing apparatus according to 3, wherein
   the update unit updates the threshold value by use of the score from which the score for the period is excluded in the history by the exclusion unit.
5. The information processing apparatus according to 3 or 4, wherein
   the estimation unit estimates an emotion of the subject by use of the score from which the score for the period is excluded in the history by the exclusion unit.
6. The information processing apparatus according to any one of 3 to 5, wherein
   the computation unit computes an average value of the score by each predetermined period, and
   the exclusion unit determines a period to be excluded by use of the average value satisfying the specific condition.
7. The information processing apparatus according to any one of 3 to 6, wherein
   the exclusion unit
      acquires schedule information of the subject, and
      determines, by use of the schedule information, a period to be excluded from a processing target.
8. The information processing apparatus according to any one of 1 to 7, wherein
   the score includes a plurality of scores indicating a plurality of types of mental states, respectively.
9. The information processing apparatus according to 8, wherein
   the estimation unit
      plots values of scores indicating two types of mental states of the subject on a coordinate as values of two coordinate axes, respectively, and
      estimates that an emotion being relevant to a region where the plotted value exists is an emotion of the user, in a case where each of four regions acquired by quartering by the coordinate axis in a region exceeding the threshold value is determined as a range of four different emotions of the subject on the coordinate, and the plotted value exists in the region exceeding the threshold value.
10. The information processing apparatus according to any one of 1 to 9, further including
   an acquisition unit that acquires biometric information from a wearable terminal worn by the subject, wherein
   the computation unit uses the biometric information acquired by the acquisition unit.
11. The information processing apparatus according to any one of 1 to 10, further including
   an acquisition unit that acquires biometric information from an external storage unit, wherein
   the computation unit uses the biometric information acquired by the acquisition unit.
12. The information processing apparatus according to any one of 1 to 11, further including
   an output processing unit that causes an external terminal to output a notification in a case where an estimation result of the emotion satisfies a criterion.
13. An information processing method including,
   by one or more computers:
   processing biometric information of a subject, and thereby computing a score indicating mental state of the subject;
   estimating an emotion of the subject by use of a criterion including a threshold value associated with the subject, and the score; and
   updating the threshold value by use of a history of the score.
14. The information processing method according to 13, including,
   by the one or more computers,
   setting the threshold value of the subject by use of a maximum value and a minimum value of the score of the subject.
15. The information processing method according to 13 or 14, further including,
   by the one or more computers,
   excluding the score for a period satisfying a specific condition in the history of the score.
16. The information processing method according to 15, including,
   by the one or more computers,
   updating the threshold value by use of the score from which the score for the period is excluded in the history.
17. The information processing method according to 15 or 16, including,
   by the one or more computers,
   estimating an emotion of the subject by use of the score from which the score for the period is excluded in the history.
18. The information processing method according to any one of 15 to 17, including,
   by the one or more computers:
   computing an average value of the score by each predetermined period; and
   determining a period to be excluded, by use of the average value satisfying the specific condition.
19. The information processing method according to any one of 15 to 18, including,
   by the one or more computers:
   acquiring schedule information of the subject; and
   determining, by use of the schedule information, a period to be excluded from a processing target.
20. The information processing method according to any one of 13 to 19, wherein
   the score includes a plurality of scores indicating a plurality of types of mental states, respectively.
21. The information processing method according to 20, including,
   by the one or more computers:
   plotting values of scores indicating two types of mental states of the subject on a coordinate as values of two coordinate axes, respectively; and
   estimating that an emotion being relevant to a region where the plotted value exists is an emotion of the user, in a case where each of four regions acquired by quartering by the coordinate axis in a region exceeding the threshold value is determined as a range of four different emotions of the subject on the coordinate, and the plotted value exists in the region exceeding the threshold value.
22. The information processing method according to any one of 13 to 21, further including,
   by the one or more computers:
   acquiring biometric information from a wearable terminal worn by the subject; and
   computing the score by use of the acquired biometric information.
23. The information processing method according to any one of 13 to 22, further including,
   by the one or more computers:
   acquiring biometric information from an external storage unit; and
   computing the score by use of the acquired biometric information.
24. The information processing method according to any one of 13 to 23, further including,
   by the one or more computers,
   causing an external terminal to output a notification in a case where an estimation result of the emotion satisfies a criterion.
25. A computer-readable program medium storing a program for causing a computer to execute:
   a procedure of processing biometric information of a subject, and thereby computing a score indicating a mental state of the subject;
   a procedure of estimating an emotion of the subject by use of a criterion including a threshold value associated with the subject, and the score; and
   a procedure of updating the threshold value by use of a history of the score.
26. The computer-readable program medium according to 25 storing the program for causing the computer to execute,
   in the procedure of updating, setting the threshold value of the subject by use of a maximum value and a minimum value of the score of the subject.
27. The computer-readable program medium according to 25 or 26 storing the program for causing the computer to further execute,
   a procedure of excluding the score for a period satisfying a specific condition in the history of the score.
28. The computer-readable program medium according to 27 storing the program for causing the computer to execute,
   in the procedure of updating, updating the threshold value by use of the score from which the score for the period is excluded in the history by the procedure of excluding.
29. The computer-readable program medium according to 27 or 28 storing the program for causing the computer to execute,
   in the procedure of estimating, estimating an emotion of the subject by use of the score from which the score for the period is excluded in the history by the procedure of excluding.
30. The computer-readable program medium according to 27 to 29 storing the program for causing the computer to execute:
   in the procedure of computing, computing an average value of the score by each predetermined period; and,
   in the procedure of excluding, determining a period to be excluded, by use of the average value satisfying the specific condition.
31. The computer-readable program medium according to any one of 27 to 30 storing the program for causing the computer to execute,
   in the procedure of excluding,
      acquiring schedule information of the subject, and
      determining, by use of the schedule information, a period to be excluded from a processing target.
32. The computer-readable program medium according to any one of 25 to 31, wherein
   the score includes a plurality of scores indicating a plurality of types of mental states, respectively.
33. The computer-readable program medium according to 32 storing the program for causing the computer to execute,
   in the procedure of estimating,
      plotting values of scores indicating two types of mental states of the subject on a coordinate as values of two coordinate axes, respectively, and
      estimating that an emotion being relevant to a region where the plotted value exists is an emotion of the user, in a case where each of four regions acquired by quartering by the coordinate axis in a region exceeding the threshold value is determined as a range of four different emotions of the subject on the coordinate, and the plotted value exists in the region exceeding the threshold value.
34. The computer-readable program medium according to any one of 25 to 33 storing the program for causing the computer to further execute:
   a procedure of acquiring biometric information from a wearable terminal worn by the subject; and,
   in the procedure of computing, computing the score by use of the biometric information acquired in the procedure of acquiring.
35. The computer-readable program medium according to any one of 25 to 34 storing the program for causing the computer to further execute:
   a procedure of acquiring biometric information from an external storage unit; and,
   in the procedure of computing, computing the score by use of the biometric information acquired in the procedure of acquiring.
36. The computer-readable program medium according to any one of 25 to 35 storing the program for causing the computer to execute,
   a procedure of causing an external terminal to output a notification in a case where an estimation result of the emotion satisfies a criterion.
37. A program for causing a computer to execute:
   a procedure of processing biometric information of a subject, and thereby computing a score indicating a mental state of the subject;
   a procedure of estimating an emotion of the subject by use of a criterion including a threshold value associated with the subject, and the score; and
   a procedure of updating the threshold value by use of a history of the score.
38. The program according to 37 for causing the computer to execute,
   in the procedure of updating, setting the threshold value of the subject by use of a maximum value and a minimum value of the score of the subject.
39. The program according to 37 or 38 for causing the computer to further execute,
   a procedure of excluding the score for a period satisfying a specific condition in the history of the score.
40. The program according to 39 for causing the computer to execute,
   in the procedure of updating, updating the threshold value by use of the score from which the score for the period is excluded in the history by the procedure of excluding.
41. The program according to 39 or 40 for causing the computer to execute,
   in the procedure of estimating, estimating an emotion of the subject by use of the score from which the score for the period is excluded in the history by the procedure of excluding.
42. The program according to any one of 39 to 41 for causing the computer to execute:
   in the procedure of computing, computing an average value of the score by each predetermined period; and,
   in the procedure of excluding, determining a period to be excluded, by use of the average value satisfying the specific condition.
43. The program according to any one of 39 to 42 for causing the computer to execute,
   in the procedure of excluding,
      acquiring schedule information of the subject, and
      determining, by use of the schedule information, a period to be excluded from a processing target.
44. The program according to any one of 37 to 43, wherein
   the score includes a plurality of scores indicating a plurality of types of mental states, respectively.
45. The program according to 44 for causing the computer to execute,
   in the procedure of estimating,
      plotting values of scores indicating two types of mental states of the subject on a coordinate as values of two coordinate axes, respectively, and
      estimating that an emotion being relevant to a region where the plotted value exists is an emotion of the user, in a case where each of four regions acquired by quartering by the coordinate axis in a region exceeding the threshold value is determined as a range of four different emotions of the subject on the coordinate, and the plotted value exists in the region exceeding the threshold value.
46. The program according to any one of 37 to 45 for causing the computer to further execute:
   a procedure of acquiring biometric information from a wearable terminal worn by the subject; and,
   in the procedure of computing, computing the score by use of the biometric information acquired in the procedure of acquiring.
47. The program according to any one of 37 to 46 for causing the computer to further execute:
   a procedure of acquiring biometric information from an external storage unit; and,
   in the procedure of computing, computing the score by use of the biometric information acquired in the procedure of acquiring.
48. The program according to any one of 37 to 47 for causing the computer to execute,
   a procedure of causing an external terminal to output a notification in a case where an estimation result of the emotion satisfies a criterion.

### Reference Signs List

1 Information processing system
3 Communication network
50 Wearable terminal
60 User terminal
70 Server apparatus
80 Manager terminal
100 Information processing apparatus
102 Computation unit
104 Estimation unit
106 Update unit
110 Acquisition unit
112 Exclusion unit
114 Output processing unit
120 Storage apparatus
130 Biometric information
140 Score history information
150 Threshold value information
1000 Computer
1010 Bus
1020 Processor
1030 Memory
1040 Storage device
1050 Input/output interface
1060 Network interface

What is claimed is:

1. An information processing apparatus comprising:
at least one memory storing instructions; and
at least one processor configured to execute the instructions to:
process biometric information of a subject, and thereby compute a score indicating a mental state of the subject;
estimate an emotion of the subject by use of a criterion and the score, the criterion including a threshold value associated with the subject; and
update the threshold value by use of a history of the score.

2. The information processing apparatus according to claim 1, wherein the at least one processor is further configured to execute the instructions to
set the threshold value of the subject by use of a maximum value and a minimum value of the score of the subject.

3. The information processing apparatus according to claim 1, wherein the at least one processor is further configured to execute the instructions to
exclude the score for a period satisfying a specific condition in the history of the score.

4. The information processing apparatus according to claim 3, wherein the at least one processor is further configured to execute the instructions to
update the threshold value by use of the score from which the score for the period is excluded in the history

5. The information processing apparatus according to claim 3, wherein the at least one processor is further configured to execute the instructions to
estimate an emotion of the subject by use of the score from which the score for the period is excluded in the history.

6. The information processing apparatus according to claim 3, wherein the at least one processor is further configured to execute the instructions to
compute an average value of the score by each predetermined period, and
determine a period to be excluded by use of the average value satisfying the specific condition.

7. The information processing apparatus according to claim 3, wherein the at least one processor is further configured to execute the instructions to
acquire schedule information of the subject, and
determine, by use of the schedule information, a period to be excluded from a processing target.

8. The information processing apparatus according to claim 1, wherein
the score includes a plurality of scores indicating a plurality of types of mental states, respectively.

9. The information processing apparatus according to claim 8, wherein the at least one processor is further configured to execute the instructions to:
plot values of scores indicating two types of mental states of the subject on a coordinate as values of two coordinate axes, respectively; and
estimate that an emotion being relevant to a region where the plotted value exists is an emotion of the user, in a case where each of four regions acquired by quartering by the coordinate axis in a region exceeding the threshold value is determined as a range of four different emotions of the subject on the coordinate, and the plotted value exists in the region exceeding the threshold value.

10. The information processing apparatus according to claim 1, wherein the at least one processor is further configured to execute the instructions to:
acquire biometric information from a wearable terminal worn by the subject; and
use the biometric information acquired.

11. The information processing apparatus according to claim 1, wherein the at least one processor is further configured to execute the instructions to:
acquire biometric information from an external storage unit; and
use the biometric information acquired.

12. The information processing apparatus according to claim 1, wherein the at least one processor is further configured to execute the instructions to
cause an external terminal to output a notification in a case where an estimation result of the emotion satisfies a criterion.

13. An information processing method comprising,
by one or more computers:
processing biometric information of a subject, and thereby computing a score indicating a mental state of the subject;
estimating an emotion of the subject by use of a criterion including a threshold value associated with the subject, and the score; and
updating the threshold value by use of a history of the score.

14-24. (canceled)

25. A non-transitory computer-readable medium storing a program for causing a computer to execute:
a procedure of processing biometric information of a subject, and thereby computing a score indicating a mental state of the subject;
a procedure of estimating an emotion of the subject by use of a criterion including a threshold value associated with the subject, and the score; and
a procedure of updating the threshold value by use of a history of the score.

26-36. (canceled)
